(19) United States
(12) Patent Application Publication
(10) Pub. No.: US 2001/0018616 A1
(43) Pub. Date: Aug. 30, 2001

(54) Device for anchoring an endoprosthesis in a bone

(76) Inventor: Jan M. Schwab, Tubingen (DE)

Correspondence Address: Harness, Dickey & Pierce, P.L.C., P. O. Box 828, Bloomfield Hills, MI, 48303, US

(21) Appl. No.: 09/750,647

(22) Filed: Dec. 28, 2000

Related U.S. Application Data
(63) Continuation of application No. PCT/EP99/04461, filed on Jun. 28, 1999.

(30) Foreign Application Priority Data
Jul. 2, 1998 (DE) 198 29 589.8

(51) Int. Cl. A61F 2/36; A61F 2/30

(52) U.S. Cl. 623/23.17; 623/23.23; 623/23.46

(57) ABSTRACT

A device is provided for anchoring an endoprosthesis in a bone, for example a thigh portion of a hip joint endoprosthesis in a femur. The device is configured in the form of a quiver-like receptacle which can be implanted in the bone for receiving a shaft of the endoprosthesis. The receptacle comprises an inner wall enclosing the shaft and an outer wall disposed at a distance therefrom. A space between the inner wall and the outer wall is filled with a medium, in which an externally applied pressure is uniformly distributed substantially to all sides for damping impacts exerted on the endoprosthesis.

### CROSS REFERENCE TO PENDING APPLICATION

This application is a continuation of pending International Application PCT/EP99/04461 filed on Jun. 28, 1999, which designates the United States.

### BACKGROUND OF THE INVENTION

The invention relates to a device for anchoring an endoprosthesis in a bone, for example a thigh portion of a hip joint endoprosthesis in a femur.

Endoprostheses are used in medicine to replace destroyed or irrepairably damaged parts of the human skeleton. In particular, endoprostheses are used as artificial replacements for joints, especially large joints such as the hip joint or the knee joint. Especially with a hip joint, the ball socket on the pelvis or the head including the neck of the femur or both can be replaced in the human skeleton. Such total endoprosthetic systems generally have a shaft which is implanted in the healthy bone structure of the skeletal part to be replaced, while the neck and the head of the shaft extend from the bone.

The thigh portion of a hip joint endoprosthesis comprises an approximately cylindrical, conical or also slightly curved shaft, which has a neck at its upper end and finally an artificial ball attached thereto. The shaft of the thigh portion of such endoprostheses are inserted into the marrow cavity of the femur, which has been previously prepared.

For younger patients with sufficient bone substance capable of growth, the endoprosthesis shaft is normally placed directly in the prepared marrow cavity, where the bone tissue next to the shaft grows onto the shaft. In older patients with complicated fractures of the corresponding skeletal parts or with unstable bone substance, the shaft of the endoprosthesis is normally cemented into the narrow cavity. In such cases, particularly for older patients, sufficient bone tissue capable of growth is no longer present to guarantee a reliable, stable anchoring of the shaft in the bone by growth of bone tissue onto the shaft.

Although considerable progress has been achieved in prosthetic surgery in the past three decades, a durable, stable anchoring of the prosthesis, free of abrasion and secured against release, has remained a problem with endoprostheses implanted with or without cement.

Such endoprostheses are subject to large loads through movement of the prosthesis patient. For example, when descending a stairs forces in the range of three to four times the body weight of the person are exerted on the thigh portion of the hip joint endoprosthesis. These forces act directly on the interface between the bone and endoprosthesis or between the cement and the endoprosthesis through the shaft.

The endoprosthesis shaft moves in the marrow cavity due to the changing loads caused by the person moving. The shaft rubs against the surface of the bone tissue or locally exerts forces on the bone tissue. Thus the growth of the bone onto the shaft is hindered or the bone tissue already grown or the cement deteriorates with time. A durable, stable anchoring of the endoprosthesis in the bone is endangered through loads or peak loads, apart from rotational forces, which act not only vertically but in transverse direction to the endoprosthesis shaft. This can cause excess loads on the bone-endoprosthesis interface or the cement-endoprosthesis interface either locally or at particular points, in particular in the region of the lower end of the shaft. The mentioned interface is subjected to changing forces even with purely vertical loads on the endoprosthesis, which in time damage the interface. This can lead to an osteologic reaction, which can result in a loosening of the endoprosthesis. Thus, a protection of the critical interface between the bone and the endoprosthesis or between the cement and the endoprosthesis is decisive for the lifetime of the artificial joint.

From U.S. Pat. No. 4,892,550 an endoprosthesis device is known which can be installed immovably in a medullary canal under a constant predetermined uniformly distributed pressure. The rigid prosthesis shaft is typically embraced by an elastomeric element designed for continuous pressurization into compressed resilient anchorage with the medullary canal surface. The tubular elastomeric element can be pressurized in various ways, such as by a captive gas, or by a fluent material until this material takes a set, or by a combination of pressurized gas and settable fluent material. The device for anchoring the shaft of the prosthesis in the bone is configured in quiver-like fashion. However, the captive gas or the fluent material is not present in the distal most end region of the receptacle, where the receptacle is made from a uniform material in one piece. Impacts vertically acting on the endoprosthesis cannot be damped sufficiently, with the risk that the endoprosthesis can axially further penetrate in the medullary canal.

The object of the invention is therefore to provide a durable, stable and non-releasable anchoring of an endoprosthesis in the bone into which it is implanted.

### SUMMARY OF THE INVENTION

According to the present invention, this object is achieved by a device for anchoring an endoprosthesis in a bone, for example, a thigh part of a hip joint endoprosthesis in a femur, wherein the device is configured in form of a quiver-like receptacle implantable in the bone for receiving a shaft of the endoprosthesis, the receptacle comprising:

an inner wall enclosing the shaft,

an outer wall disposed at a distance from the inner wall,

a medium filled in a space between the inner wall and the outer wall, in which medium an externally applied pressure is uniformly distributed substantially to all sides for damping impacts exerted on the endoprosthesis,

wherein the inner wall is disposed at a distance from the outer wall also at a distal most end of the receptacle.

The device of the present invention produces a mechanical decoupling of the shaft of the endoprosthesis from the tissue of the bone in which the endoprosthesis is implanted. With the device of the present invention, a particularly harmful momentum transfer from the shaft to the adjacent bone tissue or the cement is avoided. The shaft of the endoprosthesis is received in the quiver-like receptacle and lies against the inner wall of the receptacle. The separate outer wall of the quiver-like receptacle lies against the bone or the cement. When the endoprosthesis is subjected to a load, the resulting forces are dampened by the medium present between the inner wall and the outer wall of the receptacle, also at the distal most end between bottom portions of the outer and the inner wall, for example, dissipated. These forces are transmitted to the bone tissue or the cement adjacent to the outer wall, but without strong impact or with distinctly reduced and distributed momentum. The device according to the present invention thus acts as a damping buffer or a shock absorber between the shaft of the endoprosthesis and the adjacent bone tissue or cement. The outer wall which is in connection with the cement or the tissue of the bone is not disturbed in this manner, so that no abrasion or friction occurs between the outer wall and the adjacent cement or the bone tissue of the bone. The interface created between the outer wall of the receptacle and the cement or tissue is thus guarded against the alternating or fluctuating forces which act on the endoprosthesis.

A medium may be present between the inner wall and the outer wall, by which an externally applied pressure is uniformly distributed to all sides. For example, when using a fluid as the medium, a force acting on the inner wall of the receptacle, in particular a local or point force, is uniformly distributed in the receptacle. Local load peaks at the interface between the outer wall and the bone or cement are avoided. Preferably, the medium is composed such that it possesses the properties of damping and pressure distribution.

The present invention therefore provides a durable, stable anchoring of an endoprosthesis in a bone, because the previous rigid connection between the shaft and the bone or cement is avoided. The object underlying the invention is therefore completely achieved.

In a preferred embodiment, the inner wall comprises a slight elastic movability.

The feature has the advantage that the impact forces acting on the endoprosthesis are already pre-dampened by the inner wall. Slightly elastically movable will be understood in that the freedom of movement of the endoprosthesis is not substantially larger than if the endoprosthesis -were implanted in the bone without the quiver-like receptacle, i.e. the endoprosthesis is not float-mounted in the receptacle, but is securely anchored. The momentum transfer to the medium resulting from an elastic movement of the inner wall is damped by the medium and/or uniformly distributed in the medium.

In a further preferred embodiment, the outer wall is configured to be rigid.

The feature has the advantage that the outer wall provides a secure anchoring of the receptacle and thus the endoprosthesis in the bone.

In a further preferred embodiment, the inner wall and/or the medium has a form memory.

The feature has the advantage that the inner wall always returns to its original form after an elastic movement due to a force acting on the endoprosthesis shaft, so that the endoprosthesis does not shift with time with respect to the bone in which it is implanted.

In a further preferred embodiment, additional walls are formed in the space between the inner wall and the outer wall, which are connected to the inner wall and the outer wall and subdivide the space into two or more chambers.

The additional walls contribute in advantageous manner to the increased stability of the quiver-like receptacle.

Preferably, the additional walls are elastically movable.

The feature has the advantage that the further walls contribute to the dampening of shocks or impacts in addition to the medium, whereby the effect of the receptacle as a shock absorber is further improved.

In a further preferred embodiment, the medium comprises a liquid.

A liquid as the medium advantageously produces a uniform distribution to all sides of a pressure exerted by the shaft on the inner wall. The further advantage is that regardless of the direction of the force acting on the shaft of the endoprosthesis, a pressure is directed towards all sides perpendicular to the outer wall and the inner wall. No forces act on the outer wall parallel to the bone tissue surface. Thus a slipping or rubbing of the outer wall with respect to the surface of the bone tissue is avoided.

A further advantage of a liquid is that an increased pressure instantaneously arises even with a small volume reduction of the space between the inner wall and the outer wall due to an impact on the endoprosthesis. The increased pressure also acts on the inner wall of the receptacle and the receptacle is then forced more securely against the shaft of the endoprosthesis. The device thus becomes self-stabilising. A liquid with a high coefficient of friction is preferred, because such a liquid is capable of dissipating kinetic energy through friction.

In a further preferred embodiment, the medium comprises a gas.

The use of a gas as the medium has the advantage that it has the property of compressibility. With a corresponding prepressurisation of the gas in the space between the inner wall and the outer wall, a dampening of impact forces is produced by a lesser or greater increase in pressure depending on the prepressurisation of the gas. A uniform pressure distribution to all sides is provided in a gas, as in a liquid.

In a further preferred embodiment, the medium is a highly viscous, elastically deformable mass or a solid material having an elastic compressibility.

The mentioned substances represent further advantageous examples of a medium, which is suited to dampen impact forces through dissipation or to cause a uniform pressure distribution in the space between the inner wall and the outer wall.

In a further preferred embodiment, a portion of the inner wall supporting a distal end of the shaft comprises a reinforcement.

This feature has the advantage that this region of the inner wall can withstand high loads over long duration, since most of the impact forces acting on the endoprosthesis are directed vertically downwardly and are transferred from the lower end of the shaft to this region of the inner wall lying below.

In a further preferred embodiment, the inner wall consists of a material containing a rubber mixture of high hardness. This feature has the advantage that the inner wall has a slightly elastic movability and on the other hand a sufficiently high rigidity and wear resistance.

In a further preferred embodiment, the inner wall comprises a metal mesh.

The feature has the advantage that the inner wall is configured with sufficiently high strength, in particular tear resistance, where on the other hand, a mesh structure still allows a sufficiently elastic movability of the inner wall. In particular, the metal mesh can still be coated with the mentioned high strength rubber mixture or a comparable coating.

In a further preferred embodiment, the outer wall consists of metal.

The feature has the advantage that the outer wall can be produced with sufficiently high strength and rigidity to achieve a secure anchoring of the receptacle in the bone.

In a further preferred embodiment, the medium is prepressurised.

The feature has the advantage that the damping characteristic of the quiver-like receptacle can be variably adjusted by the corresponding pressurisation. It is thus possible to adapt the damping characteristic of the receptacle to the osteological, anatomical conditions of the individual patient.

Preferably, the medium is prepressurised by inserting the shaft into the receptacle.

The feature has the advantage of achieving pressurisation in the medium in simple manner. For example, when using a liquid or gas as the medium, this can be accomplished in that the inner diameter of the receptacle when the shaft is not inserted is smaller than the outer diameter of the shaft. A volume reduction in the space between the inner wall and the outer wall of the receptacle results when inserting the shaft into the receptacle, which then leads to an increase in pressure in the space. The shaft is also securely held in the receptacle in a type of press-fitting.

In a further preferred embodiment, the prepressurisation is adapted to the hardness of the bone.

The feature has the advantage that the elasticity properties of the receptacle on the whole can be adjusted such that they correspond to the elasticity of the bone.

In a further preferred embodiment, a contour of the outer wall of the receptacle is adapted to a predetermined contour of an implantation region in the bone.

This feature is of particular advantage when the implantation region in the bone, for example the marrow cavity in the femur, cannot be be preprepared to form a uniform and flat bone tissue surface due to strong deterioration or due to a complicated fracture.

In a further preferred embodiment, a stent for receiving the receptacle is additionally provided, which can be implanted in the bone.

The feature has the advantage that the placement of the arrangement comprising the shaft and quiver-like receptacle in the bone is simplified. For implantation of the endoprosthesis the stent, which advantageously can be self-expandable, is placed in contracted condition in the bone, for example in a previously prepared marrow cavity. It is then released and expands to lie adjacent to the bone surface. The arrangement comprising the endoprosthesis and the quiver-like receptacle can then be inserted into the stent.

In a further preferred embodiment, the endoprosthesis is secured to the outer wall against vertical tensile forces by a tension anchor.

The feature has the advantage that a withdrawal of the endoprosthesis from the receptacle is reliably avoided when a tensile force is present in the hip region. The tension anchor can advantageously be an elastic cable with small elongation or a chain, to be able to dampen peak loads of tensile force.

In a further preferred embodiment, a bottom portion of the outer wall of the receptacle comprises an elastic shock absorbing element.

The feature has the further advantage that particularly hard impacts or shocks acting on the endoprosthesis are absorbed and damped when the endoprosthesis is forced downwardly.

In a further preferred embodiment, the device comprises means for fixing the endoprosthesis in the receptacle against rotation.

The feature has the further particular advantage that the endoprosthesis is secured in the receptacle against rotation caused by torques acting on the endoprosthesis. This is of particular importance because such endoprostheses generally are not rotationally symmetrical with respect to the axis of the shaft, and the neck of the endoprosthesis extends at an angle from the shaft. A securement against rotation according to the invention therefore has the advantage that the angular orientation of the endoprosthesis provided at the time of implantation remains unchanged.

Preferably, the means for fixing against rotation have properties for damping rotation.

The feature has the advantage that torques acting as impacts or short pulses on the endoprosthesis can be elastically dampened.

In a further preferred embodiment, the means for fixing the endoprosthesis against rotation comprise at least one strip element extending between the outer wall and the inner wall in axial direction of the receptacle and at least one rigid rib engaging the strip element, which is fixidly secured to the shaft of the endoprosthesis.

Of advantage is that a securement against rotation of the endoprosthesis in the receptacle is achieved by technically simple means.

In addition, the strip element is preferably made of hard rubber or is configured as a hollow body filled with a polymer fluid of high viscosity.

In this embodiment of at least one strip element, a securement against rotation is provided in anchoring the endoprosthesis and on the other hand, rotational damping properties are provided.

In a further preferred embodiment, the means for fixing against rotation comprise two diametrically opposite strip elements.

A reliable securement against rotation of the endoprosthesis is achieved by the simple construction of two diametrically opposite strip elements.

In a further preferred embodiment, the at least one strip element extends from about the upper end of the inner wall to the lower end of the inner wall.

A particularly stable and twist-free securement against rotation is advantageously achieved with this feature. With the provision that the at least one strip element extends only to the lower end of the inner wall, it is achieved that the medium located between the inner wall and the outer wall can still uniformly distribute and dampen an impact on the endoprosthesis throughout the entire space between the inner wall and the outer wall.

In a further preferred embodiment, the inner wall of the receptacle is not round in cross-section and the shaft of the endoprosthesis has a complementary cross-section thereto.

A securement against rotation of the endoprosthesis in the receptacle is achieved by constructively simple means through this configuration of the inner wall of the receptacle and the shaft.

In further preferred embodiments, the mentioned cross-section is approximately oval or approximately rectangular.

These forms of the cross-section can be manufactured in advantageous manner with little technical complexity both for the inner wall of the receptacle and for the endoprosthesis.

In a further preferred embodiment, the outer wall of the receptacle is not round in cross-section.

The feature has the considerable advantage that the receptacle itself is secured against rotation in the femur. Preferably, the cross-section is approximately rectangular.

Further advantages will become apparent from the following description and the attached drawings. It will be understood that the above-mentioned features and those to be discussed below are not only applicable in the given combinations, but may also be taken in other combinations or alone without departing from the scope of the present invention.

Embodiments of the invention are illustrated in the drawings and are discussed in more detail below.

### BRIEF DESCRIPTION OF THE DRAWINGS

FIG. 1 shows a device for anchoring an endoprosthesis in a femur in longitudinal cross-section according to a first embodiment.

FIG. 2 shows a device for anchoring the endoprosthesis of FIG. 1 in a femur in longitudinal cross-section according to a second embodiment of the present invention.

FIG. 3 shows a cross-section of the anchoring of an endoprosthesis according to a third embodiment, where the endoprosthesis is not shown.

FIG. 4 shows a cross-section through the device of FIG. 3 along the line IV-IV in FIG. 3.

FIG. 5 shows a perspective view of a central section of a further embodiment of the device for anchoring an endoprosthesis in the femur.

FIG. 6 shows a cross-section along the line VI-VI in FIG. 5.

FIG. 7 shows a further embodiment of a device for anchoring an endoprosthesis in the femur in an illustration corresponding to FIG. 6.

FIG. 8 shows a further embodiment of a device for anchoring an endoprosthesis in the femur in an illustration corresponding to FIG. 6.

### DETAILED DESCRIPTION OF PREFERRED EMBODIMENTS

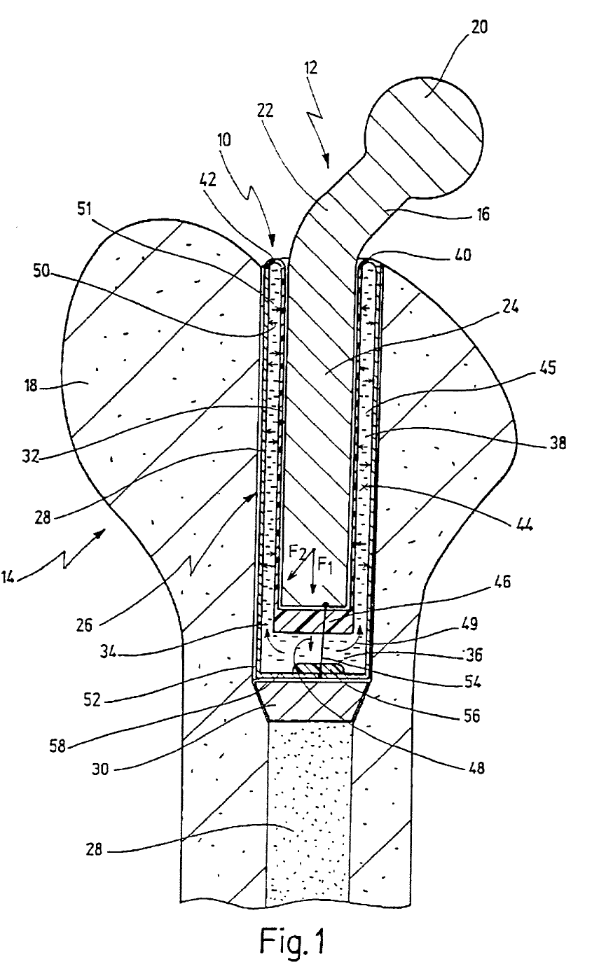

FIG. 1 shows a device for anchoring an endoprosthesis 12 in a bone 14 generally indicated by the numeral 10. The illustrated embodiment represents the endoprosthesis 12 as the thigh portion 16 of a hip joint prosthesis which is implanted in the femur 18 of a patient.

The thigh portion 16 comprises a ball 20 at its upper end, which corresponds to the ball joint in the hip of a healthy human. A neck 22 extends below the ball 20, which is followed by a shaft 24. The neck 22 is angled with respect to the shaft 24, where the ball 20 is arranged to the side of the centre axis of the shaft 24. Thus the position of the ball 20 of the thigh portion 16 corresponds to the anatomical position and orientation of the ball joint of the human hip. In the implanted condition, the neck 22 and the ball 20 project from the femur 18.

The ball 20, the neck 22 and the shaft 24 are integrally formed as one piece and are made massively of a metal suitable for medical purposes. The shaft 24 as seen in FIG. 1 is cylindrical. The present invention however is not limited to a cylindrical form of the shaft 24, but is applicable for other endoprostheses. Other endoprostheses may have a shaft extending downwardly to form a tip or may have a shaft which tapers to form a truncated cone, which in addition may also have a curved form.

The materials for the endoprosthesis 12 can be selected from Co—Cr alloys, V4A steel and ceramic or titanium alloys, for example Ti₆Al₄V.

The device 10 is configured in the form of a quiver-like receptacle 26 in which the shaft 24 of the thigh portion 16 is received. The receptacle 26 is placed in a bone marrow cavity 28 of the femur 18, where the region of the cavity in which the receptacle 26 is inserted has been operatively prepared beforehand. A conically tapered stopper 30 is placed in the cavity 28 before inserting the receptacle 26, which prevents a deeper penetration of the receptacle 26 into the cavity 28. The receptacle 26 is implanted in the cavity 28 with cement or free of cement.

The receptacle 26 is open to the top, so that the shaft 24 can be inserted in the receptacle 26 from the top. The receptacle comprises an inner wall 32, which is formed cylindrically as is the shaft 24 and which completely surrounds the shaft 24. The inner wall 32 has a U-shaped cross-section.

The receptacle 26 further comprises an outer wall 34 which when placed in the cavity 28 lies adjacent to the bone tissue of the femur 18 or, when cemented, adjacent to the cement mantle. The inner wall 32 and the outer wall 34 are securely and tightly connected to one another at the upper end 40 of the receptacle 26, where the turned over region 42 still belongs to the inner wall 32.

The inner wall 32 and the outer wall 34 define a base or bottom portion 36 as well as a leg portion 38 of the receptacle 26. In the region of the base portion 36, the outer wall 34 is further spaced from the inner wall 32 than in the region of the shank portion 38.

The inner wall 32 has a slightly elastic movability and consists of a material containing a high strength rubber mixture, where a metal net or mesh is embedded in the rubber mixture. In contrast, the outer wall 34 is rigidly formed of a metal. The outer wall 34 has the form of a cylinder closed at the bottom. The inner wall 32 also has a cylindrical contour.

A space 44 between the inner wall and the outer wall is filled with a medium 45, which is capable of damping impacts and/or is capable of uniformly distributing a pressure applied externally to substantially all sides. In the embodiment shown in FIG. 1, the space 44 is hermetically sealed by the inner wall 32 and the outer wall 34 and is completely filled with a liquid, for example a 0.9% saline solution. Other bio-compatible inert liquids can be employed, in particular those with a high inner coefficient of friction.

Instead of a liquid, the space 44 can also be filled with a gas which is provided in the space 44 with an over-pressure and depending on the given fill pressure has a certain compressibility.

A highly viscous elastically deformable mass or a solid substance having an elastic compressibility can also be placed in the space 44. The latter substances represent media which are capable of dissipating and therefore dampening mechanical energy applied in the form of impacts or kinetic energy applied variably. In addition, these substances have the ability to uniformly distribute pressure in substantially all directions.

The inner wall 32 and/or the medium 45 in the space 44 also have the property of form memory, such that a deformation of the receptacle 26 at the inner wall 32 due to an impact acting on the shaft does not remain as a deformation of the inner wall 32 or the medium 45. Rather, these elements always return to their previous form and position. In other words, the inner wall 32 and the medium 45 in the space 44 comprise a form stability due to the elastic recovery forces, without the inner wall 32 or the medium 45 having a spring effect.

A section 46 of the inner wall 32 for supporting the lower end of the shaft 24 has a reinforcement, where the reinforcement is configured such that the inner wall 32 in this region is provided with a greater thickness. A rigid plate of synthetic material or metal can also be provided in the section 46.

To implant the endoprosthesis 12, the endoprosthesis 12 is initially placed in the quiver-like receptacle 46 outside of the human body. The receptacle 26 is configured such that the clearance inner diameter of the receptacle 26 when the endoprosthesis 12 is not inserted is smaller than the outer diameter of the shaft 24. When inserting the shaft 24 in the receptacle 26, the inner wall 32 is compressed by the shaft 24 on all sides, where the volume of the space 44 is reduced. The medium contained in the space 44, for example the liquid, the gas, the highly viscous elastic mass or the elastically compressible solid is prepressurised, whereby the inner wall 32 is securely joined to the shaft 24 and the shaft 24 is seated in the receptacle 26 without play.

Subsequently, the arrangement including the endoprosthesis 12 and the receptacle 26 are placed in the bone marrow cavity 24 of the femur 18.

The effect of the device 10 will now be described when a load is applied to the endoprosthesis 12, for example a shock or impact. When a force F₁ acts on the endoprosthesis 12, such as a vertical force illustrated in FIG. 1, the lower end of the shaft 24 acts as a stamping means which exerts a pressure in the direction of the arrow 48 on the medium in the space 44 in the region of the base portion 36 of the receptacle 26. A small portion of the medium 45 is displaced out of the base portion 36 into the leg portion 38 as indicated by the arrows 49. The medium in the space 44 now causes the pressure pulse from the base portion 36 to be uniformly distributed to substantially all sides and/or to be dampened by the medium.

When the medium in the space 44 is a liquid, for example a saline solution, the effect is mainly that the pressure exerted by the shaft 24 caused by the force F is uniformly distributed in the base portion 36 and the shank portion 38 of the receptacle 26. The pressure always remains perpendicular to the inner wall 32 and the outer wall 34 as indicated by the arrows 50 and 51. Through the uniform distribution of the pressure, an excessive local or point load at an interface, for example indicated by the numeral 52, is avoided. Such an excessive load can be caused for example by a force indicated by F₂, which causes an interface overload between the outer wall 34 and the bone tissue of the bone 14.

A damping of the force or impact exerted by the shaft onto the inner wall 32 can be achieved with a medium which is capable of irreversibly transforming kinetic energy into heat due to the high inner friction of the medium. Such media for example are highly viscous liquids or masses.

The forces acting on the inner wall 32 of the receptacle 26 as indicated by the arrows 51 also produce a stabilisation of the shaft 24 in the receptacle 26. The device 10 therefore is self-stabilising.

Furthermore, the endoprosthesis 12 is secured against vertically upwardly directed forces on the receptacle 26 by a tension anchor 54. The tension anchor 54 is formed by an elastic cable or an elastic chain, whose one end is secured at the lower end of the shaft 24 of the endoprosthesis 12 and whose other end is secured at a bottom section 58 of the outer wall 34 of the receptacle 26. The tension anchor 54 prevents the endoprosthesis 12 from being drawn out of the receptacle 26 by tensile forces.

The bottom section 58 of the outer wall 34 of the receptacle 26 is also provided with an elastic shock absorbing element 56, which absorbs an impact on the endoprosthesis causing it to be pushed downwardly.

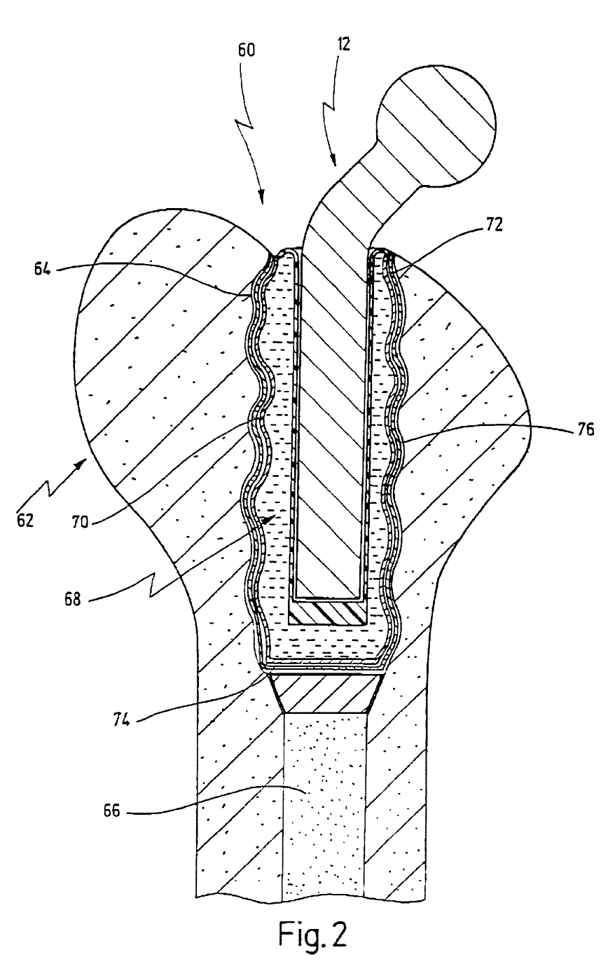

A second embodiment of a device for anchoring the endoprosthesis 12 in a bone 62 is shown in FIG. 2 and generally designated by the numeral 60. The tissue surface 64 of the bone marrow cavity 66 of the bone 62 has a disrupted structure. In such an application, where the disrupted structure of the tissue surface 64 can no longer be preprepared to form a uniform tissue surface, a quiver-like receptacle 68 is provided whose contour at an outer wall 70 of the receptacle 68 is adapted to the disrupted contour of the tissue surface 64 of the bone 62. The outer wall 70 can be fabricated on the basis of data obtained by computer tomography or obtained by means of conventional X-ray images.

In addition, a stent 72 is provided in the device 60 of FIG. 2, into which the receptacle 70 is inserted. The stent 72 comprises a bottom 74 and a peripheral outer wall 76, whereby the outer wall 76 also is adapted to the contour of the tissue surface 64. Such stents can also be used in the device 10 shown in FIG. 1, where in that case, the stent has a cylindrical form. The stent is preferably self-expandable.

When using a stent in addition to the quiver-like receptacle 26 or 68, the stent is initially inserted into the bone marrow cavity 28 or 66, where the arrangement comprising the endoprosthesis 12 and the quiver-like receptacle 26 or 68 is then inserted into the stent.

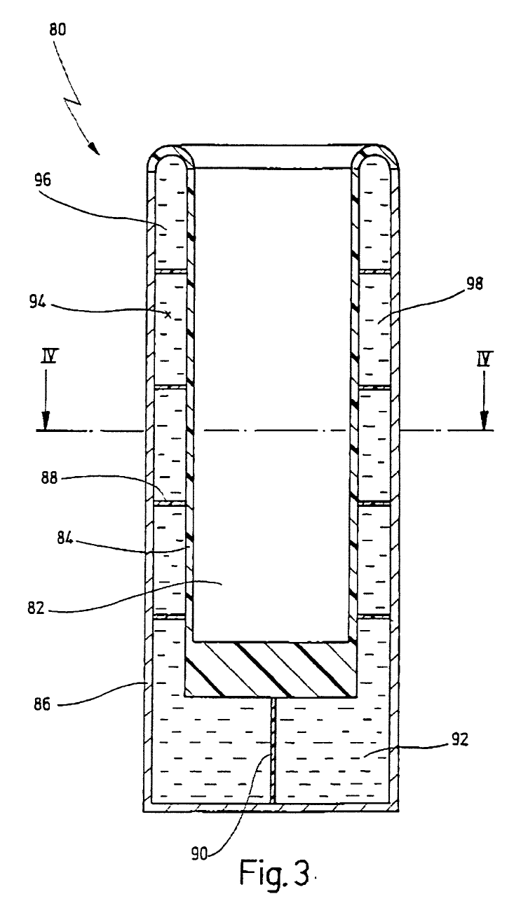

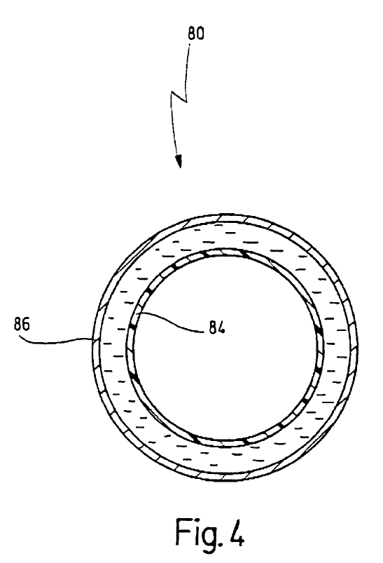

FIGS. 3 and 4 illustrate a further embodiment of a device 80 for anchoring an endoprosthesis (not shown in FIGS. 3 and 4) in a bone, which is also not shown. A quiver-like receptacle 82 differs from the receptacle 26 in FIG. 1 in that further walls 88, 90 are provided between the inner wall 84 and the outer wall 86. The further walls 88 are connected to the inner wall 84 and the outer wall 86. The walls 88 are illustrated here as horizontally arranged rings, while the wall 90 runs vertically in the base portion 92 of the receptacle 82 between the outer wall 86 and the inner wall 84.

The walls 88 and 90 are elastically displaceable. The walls 88 and 90 subdivide a space 94 between the inner wall 84 and the outer wall 86 into a number of chambers 96, where in the example of FIG. 3 a total of six chambers are present. The walls 88 and 90 can also be configured such that a medium 98 present in the space 94 can be transferred from one chamber to another. This can be accomplished for example by providing a small opening in the walls 88 or 90.

The quiver-like receptacles 26, 68, 82 can be implanted without cement or can be implanted with cement in the bones 14, 62.

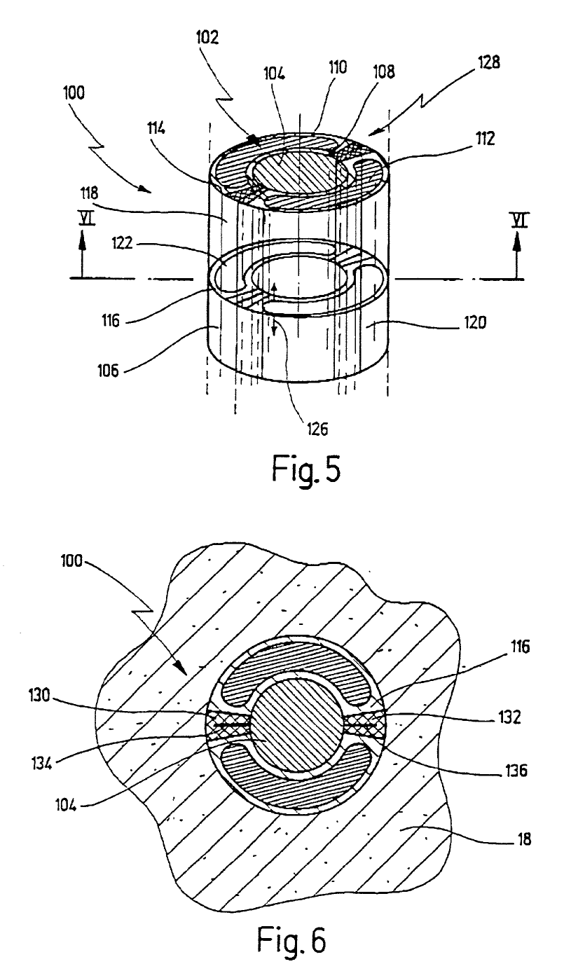

A further embodiment is shown in FIGS. 5 and 6 and illustrated as a device 100 for anchoring an endoprosthesis in the femur 18. Only a central axial section of the device 100 is shown in FIG. 5 as well as a shaft 104 of the endoprosthesis 102. The femur 18 is not illustrated in FIG. 5.

The device 100 is also configured in the form of a quiver-like receptacle 106, which as in the previous embodiments comprises an inner wall 108 and an outer wall 110. The receptacle 106 is also filled with a medium 112 between the inner wall 108 and the outer wall 110. The medium dampens impacts and/or uniformly distributes an externally applied pressure substantially to all sides, as described above in detail.

In addition, the quiver-like receptacle 106 comprises further walls 114 and 116 interconnecting the inner wall 108 and the outer wall 110, which are axially displaced from one another. Two such walls are illustrated in FIG. 5. The walls 114 and 116 run approximately horizontally and subdivide the space between the inner wall 108 and the outer wall 110 into several chambers 118, 120. Each of the walls 114, 116 comprises openings 122, 124 through which the medium 112 can communicate between the neighbouring chambers 118, 120 as indicated with the arrow 126.

The device 100 also comprises means 128 for fixing the endoprosthesis 102 against rotation in the receptacle 106. The means 128 for fixing the endoprosthesis 102 against rotation comprise two strip elements 130 extending between the inner wall 108 and the outer wall 110 in longitudinal direction of the receptacle 106. The two strip elements 130, 132 are arranged to be diametrically opposed.

The strip elements 130, 132 in cross-section form approximately a sector of a circle and extend in axial direction from the upper end (not shown in FIG. 5) of the inner wall 108 to the lower end of the inner wall 108 and pass through the horizontal walls 114, 116. Since the strip elements 130, 132 only extend to the lower end of the inner wall 108, the entire space between the inner wall 108 and the outer wall 110 form a continuous volume for the medium 112 corresponding to the embodiments of FIGS. 1 to 4. The strip element 130 and the strip element 132 consist of hard rubber or are formed as hollow bodies filled with a highly viscous polymer liquid.

The strip element 130 engages with a rigid rib 134 extending radially outwardly from the shaft 104 of the endoprosthesis 102 and extending axially over the shaft 104. The rib is fixedly connected to the shaft 104 of the endoprosthesis 102. The rib 134 is formed together with the shaft 104 or is welded to the shaft 104. A rib 136 diametrically opposed to the rib 134 also engages with the strip element 132.

The endoprosthesis 102 is thus secured in the receptacle 106 against rotation caused by rotary forces being applied to the device. Due to the constitution of the strip element 130 of a hard elastic material, such as hard rubber, the fixation is not completely stiff, but has properties of damping the rotation. In this manner, sudden torques applied to the endoprosthesis are dampened by the fixation against rotation.

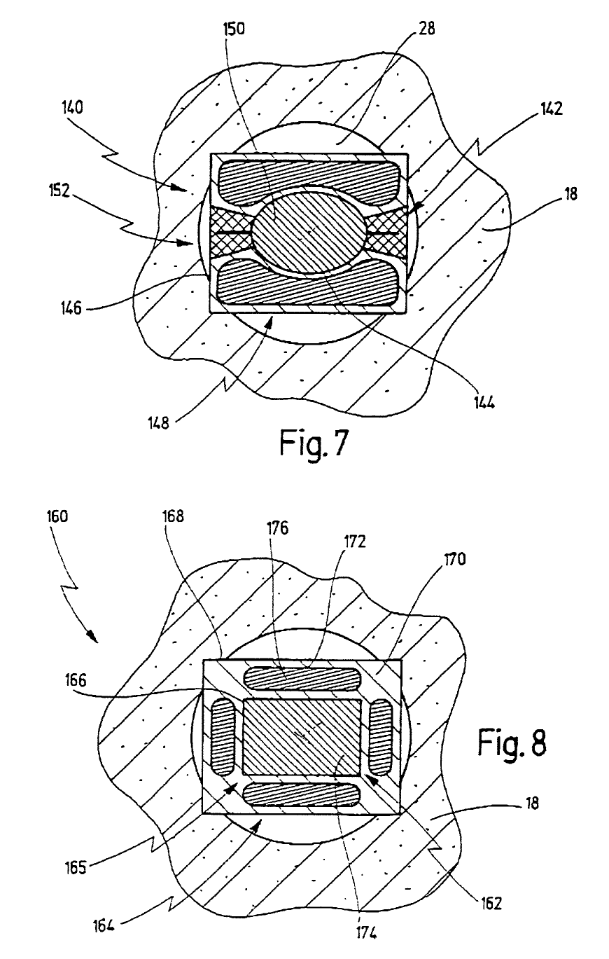

As a further embodiment of the invention, a device 140 is shown in FIG. 7 for anchoring an endoprosthesis 142 in the femur 18, which differs from the embodiment of FIGS. 5 and 6 in the geometrical configuration of an inner wall 144 and an outer wall 146 of a quiver-like receptacle 148 in which a shaft 150 of the endoprosthesis 142 is received. The inner wall 144 has an approximately oval cross-section, where the shaft 150 of the endoprosthesis 142 has a corresponding cross-sectional form adapted in form-locking manner to the form of the inner wall 144.

The outer wall 146 has an approximately rectangular form. The receptacle 148 thus secures itself in the femur 18 against rotation. In addition, the device 140 comprises means 152 for fixing the endoprosthesis 142 against rotation in the receptacle 148. The means 128 correspond to those of the embodiment in FIGS. 5 and 6 and are therefore not described in more detail here.

As can also be taken from FIG. 7, the bone marrow cavity 28 of the femur 18 has not been prepared corresponding to the geometry of the outer wall 146 with a rectangular cross-section, but still has a rounded cross-section. The receptacle 148 penetrates with its corners into the femur 18 for reliable anchoring in the femur 18. It can be expected that tissue will grow with time in the free intermediate space between the outer wall 146 of the receptacle 148 and the femur 18, which then will completely cover the outer wall 146 of the receptacle 148. The corners of the outer wall 146 can also be rounded instead of the strict right angle geometry of the outer wall 146 shown in FIG. 7.

Finally, a further embodiment of a device 160 for anchoring an endoprosthesis 162 in the femur 18 is shown in FIG. 8. The device 160 also comprises means 165 for fixing the endoprosthesis 162 against rotation in constructively very simple manner.

In contrast to the above embodiment, the device 160 is configured in the form of a quiver-like receptacle 164, whose inner wall 166 as well as an outer wall 168 is configured to be rectangular in cross-section. A shaft 174 of the endoprosthesis 162 correspondingly comprises a rectangular geometry in cross-section. With this configuration of the shaft 174 of the endoprosthesis 162 and the inner wall 166 of the receptacle 164, the endoprosthesis 162 is already anchored to be fixed against rotation in the receptacle 164, without additional measures needing to be taken.

As in the previous embodiments, the receptacle 164 includes approximately horizontally disposed walls 170 interconnecting the inner wall 166 and the outer wall 168. The walls 170 comprise four openings 172 for passage of a medium 176, which can then communicate between the chambers formed by the walls 170.

The respective walls 170 are formed with correspondingly elastic properties to achieve rotational damping of the endoprosthesis 162 in the receptacle 164.

It will be understood that the present invention, described above in conjunction with embodiments relating to the thigh portion of a hip joint prosthesis, can also be employed for the pelvic portion of a hip joint endoprosthesis or for a knee joint endoprosthesis or for endoprostheses for other large joints. It will be also understood that the features of the different embodiments can be combined with one another, for example a tension anchor according to FIG. 1 can be provided in the embodiments of FIGS. 5 to 8 or a securement against rotation of the endoprosthesis in FIGS. 5 to 8 can be provided in the device of FIG. 1. In addition, a stent can be provided in all of the embodiments, in which the quiver-like receptacle is received.

What is claimed is:

1. A device for anchoring an endoprosthesis in a bone, for example a thigh part of a hip joint endoprosthesis in a femur, wherein said device is configured in form of a quiver-like receptacle implantable in said bone for receiving a shaft of said endoprosthesis, said receptacle comprising:
an inner wall enclosing said shaft,
an outer wall disposed at a distance from said inner wall,
a medium filled in a space between said inner wall and said outer wall, in which medium an externally applied pressure is uniformly distributed substantially to all sides for damping impacts exerted on said endoprosthesis,
wherein said inner wall is disposed at a distance from said outer wall also at a distal most end of said receptacle.

2. The device of claim 1, wherein said inner wall comprises a slight elastic movability.

3. The device of claim 1, wherein said outer wall is configured to be rigid.

4. The device of claim 1, wherein at least one of said inner wall and said medium has a form memory.

5. The device of claim 1, wherein further walls are formed in said space between said inner wall and said outer wall, which are connected to said inner wall and said outer wall and subdivide said space in at least two chambers.

6. The device of claim 5, wherein said further walls are elastically movable.

7. The device of claim 1, wherein said medium comprises a liquid.

8. The device of claim 1, wherein said medium comprises a gas.

9. The device of claim 1, wherein said medium comprises a highly viscous elastically deformable mass or a solid material having an elastic compressibility.

10. The device of claim 1, wherein a portion of said inner wall supporting a distal end of said shaft comprises a reinforcement.

11. The device of claim 1, wherein said inner wall consists of a material containing a rubber mixture of high hardness.

12. The device of claim 1, wherein said inner wall comprises a metal mesh.

13. The device of claim 1, wherein said outer wall consists of a metal.

14. The device of claim 1, wherein said medium is prepressurised.

15. The device of claim 14, wherein said medium is prepressurised by inserting said shaft into said receptacle.

16. The device of claim 1, wherein said medium is prepressurised and said prepressurisation is adapted to the hardness of said bone.

17. The device of claim 1, wherein a contour of said outer wall of said receptacle is adapted to a predetermined contour of an implantation region of said bone.

18. The device of claim 1, wherein a stent is additionally provided for receiving said receptacle, which can be implanted in said bone.

19. The device of claim 1, wherein said endoprosthesis is secured against vertical tensile forces by a tension anchor on said outer wall.

20. The device of claim 1, wherein a bottom portion of said outer wall of said receptacle comprises an elastic shock absorbing element.

21. The device of claim 1, wherein said device comprises means for fixing said endoprosthesis against rotation in said receptacle.

22. The device of claim 21, wherein said means for fixing against rotation comprise properties of dampening rotation.

23. The device of claim 1, wherein said device comprises means for fixing said endoprosthesis against rotation in said receptacle and wherein said means for fixing said endoprosthesis against rotation comprise at least one strip element extending between said outer wall and said inner wall in axial direction of said receptacle and at least one rigid rib which engages in said strip element and is fixedly seated on said shaft of said endoprosthesis.

24. The device of claim 23, wherein said strip element consists of hard rubber or is formed as a hollow body filled with a highly viscous polymer fluid.

25. The device of claim 1, wherein said device comprises means for fixing said endoprosthesis against rotation in said receptacle and wherein said means for fixing against rotation comprises two strip elements diametrically opposite to one another.

26. The device of claim 24, wherein said at least one strip element extends approximately from an upper end of said inner wall to a distal end of said inner wall.

27. The device of claim 1, wherein said device comprises means for fixing said endoprosthesis against rotation in said receptacle and wherein said inner wall of said receptacle is not round in cross-section and said shaft of said endoprosthesis comprises a cross-section complementary thereto.

28. The device of claim 27, wherein said cross-section is approximately oval or approximately rectangular.

29. The device of claim 1, wherein said outer wall of said receptacle is not round in cross-section.

30. The device of claim 29, wherein said cross-section is approximately rectangular.
